(19) United States
(12) Patent Application Publication
(10) Pub. No.: US 2016/0115120 A1
(43) Pub. Date: Apr. 28, 2016

(54) METHOD FOR THE CONTROLLED HYDROFORMYLATION AND ISOMERIZATION OF A NITRILE/ESTER/OMEGA UNSATURATED FATTY ACID

(71) Applicants: ARKEMA FRANCE, Colombes (FR); UNIVERSITE DE RENNES 1, Rennes (FR); CENTRE NATIONAL DE LA RECHERCHE SCIENTIFIQUE, Paris (FR)

(72) Inventors: Jean-Luc Dubois, Millery (FR); Jean-Luc Couturier, Lyon (FR); Jeremy Ternel, Chantepie (FR); Jean-Francois Carpentier, Acigne (FR)

(21) Appl. No.: 14/896,247

(22) PCT Filed: Jun. 6, 2014
(86) PCT No.: PCT/EP2014/061899

§ 371 Date: Dec. 4, 2015

(30) Foreign Application Priority Data
Jun. 6, 2013 (FR) 13 01301

(51) Int. Cl. C07C 253/30; C07C 67/347; C07C 67/48; C07C 253/34

(52) U.S. Cl. CPC C07C 253/30; C07C 253/34; C07C 67/347; C07C 67/48

(57) ABSTRACT

A method to synthesize a fatty nitrile/ester aldehyde comprising the following steps:
1) hydroformylation of a ω-unsaturated fatty nitrile/ester/acid substrate under particular conditions of partial pressure, temperature, reaction time, conversion rate of the ω-unsaturated fatty nitrile/ester/acid reactant, catalyst, [substrate]/[metal] molar ratio and [ligand]/[metal] molar ratio so as after the reaction to obtain:
   a hydroformylation product comprising at least one fatty nitrile/ester/acid aldehyde of formula: OHC—(CH₂)ᵣ₊₂—R, and
   an isomerate comprising at least one fatty nitrile/ester/acid isomer with internal unsaturation in which at least 80% of the internal isomer(s) of the isomerate are formed of the ω-1 unsaturated isomer of formula CH₃—CH═CH—(CH₂)ᵣ₋₁—R; followed by:
2) separation and recovery of the fatty nitrile/ester/acid aldehyde and of the isomerate.

### FIELD OF THE INVENTION

The present invention is directed towards a novel method for the synthesis of fatty nitrile/ester/acid aldehydes and concerns such aldehydes that are linear meeting formula: OHC—(CH₂)ᵣ₊₂—R able to be used in industry, the polymer industry in particular such as polyamides and polyesters, said method comprising a hydroformylation step of a ω-unsaturated fatty nitrile/ester/acid.

By “ω-unsaturated fatty nitrile/ester/acid” is meant any compound of formula:

CH₂═CH—(CH₂)ᵣ—R, where R is CN or COOR₁,
R₁ being H or an alkyl radical having 1 to 4 carbon atoms,
r is an integer index such that 1≦r≦13, advantageously 2≦r≦13 and, preferably, 4≦r≦13.

### PRIOR ART

Current trends in environmental issues in the energy and chemistry sectors have led to giving priority to the use of natural raw materials from renewable resources.

For example, the polyamide industry uses a whole range of monomers formed from diamines and diacids, from lactams and from ω-amino acids. These monomers are generally manufactured via chemical synthesis using as raw materials C2 to 4 olefins, cycloalkanes or benzene, hydrocarbons derived from fossil sources. Only a few monomers are currently manufactured from bio-sourced raw materials such as castor oil allowing the manufacture of polyamide-11 marketed under the trade name Rilsan®; erucic oil allowing the manufacture of polyamide-13/13, or lesquerolic oil for the manufacture of polyamide-13.

Amongst renewable raw materials, the derivatives of fatty acids and in particular the nitriles and esters of fatty acids have a strong potential for a variety of applications. The hydroformylation of olefins using catalysts of homogeneous transition metals is a major industrial method which produces polyvalent intermediates for pharmaceutical products and fine chemicals.

However, the hydroformylation of derivatives of unsaturated fatty acids, e.g. of unsaturated fatty nitriles, remains uninvestigated. There exist efficient, selective catalytic systems for the hydroformylation of C3 to C5 olefins but they prove to be inefficient and even impossible for alkenes having a longer chain.

In addition, throughout the hydroformylation of fatty olefins a certain number of co-products are formed including isomers of internal alkenes and branched aldehydes which lead to a reduction in yields and selectivity for desired linear aldehydes.

U.S. Pat. No. 7,026,473 describes the hydroxycarbonylation or methoxycarbonylation of pentenenitrile to 5-cyanovaleric acid or its ester (6 carbon atoms) in the presence of CO (carbon monoxide) and water or alcohol respectively. Solely an example of methoxycarbonylation with methanol is given. Via reduction, the 5-cyanovaleric acid (ester) forms 6-aminocaproic acid (ester), which in turn gives ε-caprolactam by cyclisation (the monomer of Nylon-6). The method described in this document has several drawbacks. The methoxycarbonylation step is slow and costly in terms of catalyst. Conversion is not complete and requires lengthy reaction times. In addition, on account of rapid isomerization there is shifting of the double bond even if it is terminal leading to the formation of numerous co-products such as those mentioned above, in particular branched products which have to be separated from the linear product it is sought to produce. The document of patent WO97/33854 describes a method to manufacture a linear aldehyde by hydroformylation of an alkene such as hexene, butadiene, methyl 3-pentenoate or 3-pentenenitrile. This document shows that it is much more difficult to obtain a linear aldehyde (small proportion of linear products obtained) from a nitrile (3-pentenenitrile) than from an ester. Additionally, in prior art hydroformylation from a nitrile a high proportion (21%, 16.3%) of reduced product is obtained (valeronitrile), i.e. not containing any aldehyde due to hydrogenation of the double bond by the catalyst. The obtaining of linear products with these methods is detrimental to conversion. In addition, the methods described in these documents do not concern the manufacture of bio-resourced products.

The document of U.S. Pat. No. 6,307,108 describes methods to manufacture an ester aldehyde by hydroformylation of ω-unsaturated ester.

As indicated above, existing hydroformylation methods generally lead to the production of isomers of the starting reactant via isomerization of the double bond. When these isomers are recycled back to the reaction they could to a certain extent give back the starting reactant by isomerization, but they also lead to undesirable co-products, i.e. branched aldehydes by hydroformylation of the internal double bond. Also, these isomers are much less reactive than the initial ω-unsaturated compounds. As a result, if they are fully recycled back to the reaction they will gradually build up until they represent the most part of the reaction medium. In the methods described in the prior art hydroformylation is generally performed with very long reaction times of at least 20 hours to promote conversion of initial reactants and isomerization products. Productivity is therefore low. In addition the hydroformylation reaction is generally conduct in a solvent medium in particular for recovery and recycling of the catalyst (metal and ligands).

It is therefore the objective of the present invention to find a novel hydroformylation method that is easy to implement and uses renewable raw materials inasmuch as is possible.

In particular the present invention sets out to increase the productivity of the hydroformylation reaction to obtain linear aldehydes, these linear aldehydes being defined as those that are not branched, to improve product quality, reaction selectivity, to minimize the obtaining of co-products, and in particular co-products of branched aldehyde type, using the least possible amount of catalyst and therefore to target the improved general economics of the method.

It is one particular objective of the present invention to simplify the hydroformylation method applied to a ω-unsaturated fatty nitrile/ester/acid substrate and to reduce the number of steps and ingredients used whilst allowing recycling of the catalyst back to the reaction.

The Applicant has now discovered a novel method for the hydroformylation of an unsaturated fatty nitrile/ester/acid substrate under conditions allowing the avoiding of the aforementioned disadvantages, in particular by controlling parallel isomerization of said substrate which leads to the formation of co-products including branched aldehydes, the method allowing both improved productivity and selectivity as well as efficient recycling of the hydroformylation catalyst.

### DETAILED DESCRIPTION OF THE INVENTION

In the present description it is specified that when reference is made to ranges the expressions of type “ranging from . . . to . . . ” or “containing/comprising from . . . to . . . ” include the limits of the range. Conversely, expressions of the type “of between . . . and . . . ” exclude the limits of the range.

Unless otherwise indicated, percentages are expressed as molar percentages.

Unless otherwise indicated, the parameters to which reference is made are measured under atmospheric pressure.

It is also specified that in the present description a “linear aldehyde” is an aldehyde in which carbon monoxide has been added to the terminal carbon of the olefin during hydroformylation, as opposed to a “branched aldehyde” in which carbon monoxide has been added onto an internal carbon of the olefin.

Similarly, a “terminal isomer” is an isomer in which the unsaturation (double bond) is terminal, as opposed to an “internal isomer”, also called “isomer with internal unsaturation” and denoted [1-int] wherein the unsaturation is not terminal.

The subject of the present invention is therefore a method to prepare a fatty nitrile/ester aldehyde comprising the following steps:
1) hydroformylation of a ω-unsaturated fatty nitrile/ester/acid substrate selected from among the compounds of formula:

CH₂═CH—(CH₂)ᵣ—R, where R is CN or COOR₁,
R₁ being H or an alkyl radical having 1 to 4 carbon atoms,
r is an integer index such that 1≦r≦13, advantageously 2≦r≦13 and, preferably 4≦r≦13,
   wherein said substrate is reacted with carbon monoxide and dihydrogen under the following conditions:
CO partial pressure, denoted PiCO, of 40 bar or lower, preferably in the range of 5 to 20 bar, H₂ partial pressure denoted PiH₂, of 40 bar or lower, preferably in the range of 5 to 20 bar, and the ratio PiCO/PiH₂ between the respective partial pressures of CO and H₂ is in the range of 0.5:1 to 3:1,
temperature in the range of 70 to 150° C., preferably 100 to 130° C., more preferably 100 to 120° C.,
reaction time of 24 h or less,
in the presence of a catalyst comprising at least one group VIII metal, preferably at least one metal selected from among rhodium, cobalt, ruthenium, iridium and the mixtures thereof, preferably selected from among rhodium, iridium and the mixtures thereof; and at least one bidentate or monodentate ligand, preferably at least one chelating diphosphine,
[substrate]/[metal] molar ratio in the range of 5 000 to 100 000,
[ligand]/[metal] molar ratio in the range of 10:1 to 100:1,
   so as to obtain after the reaction:
a hydroformylation product comprising at least one fatty nitrile/ester/acid aldehyde of formula: OHC—(CH₂)ᵣ₊₂—R, and
an isomerate comprising at least one fatty nitrile/ester/acid isomer with internal saturation, at least 80% of the internal isomer(s) of the isomerate being formed of the ω-1 unsaturated isomer of formula CH₃—CH═CH—(CH₂)ᵣ₋₁—R; followed by:
2) separation and recovery of the fatty nitrile/ester/acid aldehyde and of the isomerate.

It is ascertained that by controlling the total % of internal olefins [1-int] obtained after the hydroformylation step, and by optimizing the distribution of these internal olefins, by regulating parameters of pressure, temperature, reaction time and use of catalyst conforming to the hydroformylation method of the invention, the method of the invention is precisely able to:
improve regioselectivity regarding linear aldehydes/branched aldehydes which are respectively denoted 2 and 3 in the reaction scheme and in the tables of the examples below: 95:5 or higher, preferably 97:3 or higher; more preferably 99:1 or higher, i.e. to reduce the obtaining of branched aldehydes 3,
   and to optimize one or more of the other following parameters:
improve chemoselectivity for hydroformylation products, denoted 2+3: 70% or higher and preferably 80% or higher;
preferably increase the conversion rate of ω-unsaturated fatty nitrile/ester/acid reactant (denoted 1 in the reaction scheme and in the tables of examples below) to end products, i.e. to hydroformylation products (2 and 3, but especially 2) and isomerate (denoted 1-int in the reaction scheme and tables of examples below), up to a value of at least 90%, a value on and after which there exists strong industrial advantage;
minimize % hydrogenation product of the double bond, the hydrogenation product being denoted 4 in the reaction scheme and tables of examples below: preferably no more than 7%, more preferably no more than 5%;
efficiently recycle the catalyst;
increase TON>100 000, TON (turnover number) being defined as the number of moles of converted ω-unsaturated fatty nitrile/ester/acid per mole of catalyst.

Step 2) to separate and recover the fatty nitrile/ester/acid aldehyde and isomerate stops shifting of the internal unsaturation of the isomers via isomerization and allows enhanced re-use of the isomerate.

Rather than recycling the internal isomers [1-int] in the hydroformylation reaction (hereafter “HF”), or lengthening the hydroformylation reaction time to allow the catalyst to isomerize the internal isomers to terminal isomers, and then hydroformylating to linear aldehyde, the method of the invention implements the hydroformylation step with gains in productivity translating as a hydroformylation step which takes place in the shortest time possible and sufficient to obtain complete or near-complete conversion (preferably >90%, even 100%). With the method of the invention it is possible to avoid recycling, to halt isomerization of the isomers, to reduce the obtaining of hydrogenation product and thereby improve selectivity for linear aldehyde.

In fact the technical solution of the method of the invention is to operate with high (even complete) conversion of initial reactant without however seeking to convert the isomerization products of this initial reactant, high conversion meaning a conversion rate of at least 90%.

Unexpectedly, it is the control over synthesis of the isomers, and in particular the halting of their isomerization through recovery thereof after the hydroformylation step, which allows slowing of the reaction to be avoided, the HF catalyst to be efficiently recycled and the productivity and selectivity of the HF reaction to be increased.

The initial omega-unsaturated reactant:
is partly converted by hydroformylation to fatty nitrile/ester/acid aldehyde that is essentially linear 2 (preferably containing no more than 5% of branched compounds 3), and
partly converted to 1-int isomers characterized by limited (controlled) shifting of the double bond towards internal positions, the internal isomers comprising at least 80% of ω-1 isomers.

In one particularly advantageous variant of the invention, the ω-unsaturated fatty nitrile/ester/acid substrate meets formula CH₂═CH—(CH₂)ᵣ—R, with R═COOR₁, R₁ being H or an alkyl radical having 1 to 4 carbon atoms.

In the present description, “ω-x” indicates the position of the first unsaturation starting from the side opposite the nitrile, ester or acid R group.

By “isomerate” in the meaning of the invention is meant at least one isomer with internal unsaturation (ω-x: ω-1, ω-2, ω-3 . . . ) of the ω-unsaturated fatty nitrile/ester/acid, which has a terminal unsaturation, the said isomerate possibly also containing the initial non-converted substrate, i.e. the ω-unsaturated fatty nitrile/ester/acid. The internal isomers of the isomerate may be cis and/or trans.

By “unsaturated fatty nitrile/ester/acid” in the meaning of the present invention is to be understood unsaturated fatty nitrile or ester or acid, i.e. unsaturated fatty nitrile or unsaturated fatty ester or unsaturated fatty acid.

By “ω-unsaturated fatty nitrile/ester/acid substrate” is meant an “unsaturated fatty nitrile/ester/acid” of which at least 90% comprises a terminal “ω” unsaturation, the said substrate possibly comprising no more than 10% of ω-1 unsaturated fatty nitrile/ester/acid, i.e. no more than 10% of “unsaturated fatty nitrile/ester/acid” having an internal ω-1 unsaturation. In the event that the “ω-unsaturated fatty nitrile/ester/acid substrate” should comprise saturated compounds, the latter not reacting during the hydroformylation reaction, these must be removed after his reaction as must be the hydrogenation product of the double bond 4.

The starting unsaturated fatty nitrile used in the method of the invention is generally obtained from unsaturated (or hydroxylated) fatty acid or ester compounds by nitrilation (ammoniation) of at least one acid or ester function of these compounds which may be derived from raw materials of fossil origin or from renewable sources.

The unsaturated fatty acid or ester compounds can be obtained for example using the method described in the document of U.S. Pat. No. 4,510,331. This document particularly describes the preparation of 7-octenoic acid by isomerization of 2,7-octadien-1-ol to 7-octen-1-al, followed by oxidization of the latter to acid. 2,7-octadien-1-ol is produced industrially by reaction (“telomerization”) of butadiene with water in the presence of palladium catalyst in accordance with the method described in the documents of patents GB2074156A and DE3112213. This type of method uses raw materials of fossil origin.

Alternatively, unsaturated fatty nitriles are produced from unsaturated fatty acids or esters of renewable origin, derived from natural oils. These methods recently developed by Arkema are notably described in the documents of patents: WO2010055273, FR11.55174, FR11.56526 and FR11.57542.

By unsaturated fatty nitrile in the meaning of the invention is preferably meant those obtained at least in part from natural unsaturated fatty acids.

Said unsaturated fatty nitrile can be obtained in particular from an unsaturated fatty acid (or ester) of natural origin of formula:

(R′—CH═CH—[(CH₂)_{q}—CH═CH]ₘ—(CH₂)ₙ—COO—)ₚ-G
wherein: R′ is H, an alkyl radical having 1 to 11 carbon atoms optionally containing a hydroxyl function,
   q equals 0 or 1,
   m is an integer index in the range of 0 to 5 and preferably 0 to 2,
   n is an integer index in the range of 2 to 13,
   p is an integer index such that 1≦p≦3, and
   G is H (a hydrogen), an alkyl radical having 1 to 11 carbon atoms or a radical having 2 to 3 carbon atoms carrying 1 or 2 hydroxyl functions,
   the double bond(s) C═C possibly being of cis or trans conformation, said preparation comprising the ammoniation (action whereby ammonia is added to a product) of the carbonyl function of the unsaturated fatty acid (or ester) of natural origin to a nitrile function.

The reaction scheme of the synthesis of nitriles from acids via ammoniation (or nitrilation, the two terms being used indifferently) well known to persons skilled in the art can be summarized as follows:

R—COOH+NH₃→[R—COO—NH₄⁺]→[R—CONH₂]+H₂O→RCN+H₂O

This scheme applies both to natural fatty acids (esters) and to ω-unsaturated fatty acids. The method can be a batch method in liquid or gas phase or a continuous method in gas phase. The reaction is conducted at high temperature, higher than 250° C. and in the presence of catalyst which is generally a metal oxide and most frequently zinc oxide. The continuous removal of the water formed also carries off the ammonia which has not reacted and allows rapid completion of the reaction.

Ammoniation in liquid phase is well adapted for long fatty chains (having at least 10 carbon atoms). However, when operating with shorter chain lengths, ammoniation in gas phase may become more suitable. It is also known from GB 641,955 to conduct ammoniation using urea or cyanuric acid as agent. Any other ammonia source may also be used.

According to one particular embodiment, the unsaturated fatty nitrile used in the invention is produced from natural long chain unsaturated fatty acids. By “natural long chain fatty acid” is meant an acid derived from the vegetable or animal kingdom including algae or other micro-organisms, and hence renewable, having 6 to 24 carbon atoms, preferably having at least 7 carbon atoms (if the final amino acid has at least 8 C), more preferably at least 8 carbon atoms, further preferably at least 10 carbon atoms and still further preferably at least 14 carbon atoms per molecule. These various acids are derived from vegetable oils extracted from various plants such as sunflower, rapeseed, camelina, castor, lesquerella, olive, soybean, palm, coriander, celery, dill, carrot, fennel, Limnanthes Alba (meadowfoam). They are also derived from the animal world, whether land or marine and in the latter case both in the form of fish and mammals as well as algae. In general it is in the form of fats from ruminants, fish such as cod or sea mammals such as whales or dolphins.

As unsaturated fatty acid more particularly suitable for implementing the invention, mention can be made of the following: petroselenic acid (cis-6-octadecenoic acid), its derivative 6-heptenoic acid obtained by ethenolysis (cross metathesis with ethylene), a-linolenic acid (6-9-12-octadecatrienoic), these acids able to be obtained from coriander for example; cis-8-eicosenoic acid, cis-5,8,11,14-eicosatrienoic acid (arachidonic acid), ricinoleic acid which after dehydration gives conjugated 8,10-octadecadienoic acid; caproleic acid (cis-9-decenoic), palmitoleic acid (cis-9-hexadecenoic), myristoleic acid (cis-9-tetradecenoic), oleic acid (cis-9-octadecenoic), 9-decenoic acid obtained by ethenolysis of an oleic acid for example, elaidic acid (trans-9-octadecenoic), ricinoleic acid (12-hydroxy-cis-9-octadecenoic), gadoleic acid (cis-9-eicosenoic), linoleic acid (9-12-octadecadienoic), rumenic acid (9-11-octadecadienoic), conjugated linoleic acid (9-11-octadecadienoic), these acids able to be obtained from sunflower seed, rapeseed, castor, olive, soybean, palm, flax, avocado, sea-buckthorn, coriander, celery, dill, carrot, fennel, Limnanthes (meadowfoam); 10-12 conjugated linoleic acid (10-12-octadecadienoic), 10-undecylenic acid obtained by thermal cracking of the methyl ester of ricinoleic acid for example; vaccenic acid (cis-11-octadecenoic), gondoic acid (cis-11-eicosenoic), lesquerolic acid (14-hydroxy-cis-11-eicosenoic), cetoleic acid (cis-11-docosenoic) able to be obtained from Lesquerella oil (lesquerolic), Camelina sativa oil (gondoic), oil from a plant of the Sapindaceae family, from fish fat, oil from microalgae (cetoleic) by dehydration of 12-hydroxystearic acid itself obtained by hydrogenation of ricinoleic acid (vaccenic acid and its trans equivalent), of conjugated linioleic acid (9-11-octadecadienoic), obtained for example by dehydration of ricinoleic acid; 12-octadecenoic acid (cis or trans) obtained for example by dehydration of 12-hydroxystearic aid (abbreviated 12HSA) itself obtained by hydrogenation of ricinoleic acid, 10-12 conjugated linoleic acid (10-12-octadecadienoic), 12-tridecenoic acid obtained by thermal cracking of the ester (methyl ester in particular) of lesquerolic acid; erucic acid (cis-13-docosenoic) and brassidic (trans-13-docosenoic) which can be obtained for example from erucic rapeseed, Lunaria or Crambe Maritima; 13-eicosenoic acid (cis or trans) obtained by dehydration of 14-hydroxyeicosanoic acid itself obtained by hydrogenation of lesquerolic acid, 14-eicosenoic acid (cis or trans) obtained by dehydration of 14-hydroxyeicosanoic acid (abbreviation 14HEA) itself obtained by hydrogenation of lesquerolic acid (dehydration can be conducted on both sides of OH), nervonic acid (cis-15-tetracosoic) which can be obtained from Melania oleifera and Lunaria (lunaria annua also known under the name of silver dollar, money plant, honesty); or the mixtures thereof. It is also possible to omit the dehydration step of the 12HSA and 14HEA acids by conducting conversion to nitrile directly on these saturated, hydroxylated fatty acids as described in the document of the patent filed under number FR11.56526. One advantage of this solution is that the hydrogenation of ricinoleic acid in a mixture with the other fatty acids of castor oil leads to a mixture that only contains 12HSA acid, stearic acid and palmitic acid as majority species. Dehydration following (or simultaneously with) conversion to nitrile leads to a very clean nitrile containing more than 85% of monounsaturated nitrile. The same applies to 14HEA, as described in the document of patent FR11.56526.

Amongst the aforementioned unsaturated fatty acids, preference is given to those which are most abundantly available and in particular unsaturated fatty acids at position δ-9 or δ-10 with numbering starting from the acid group. Preference is effectively given to the use of fatty nitriles and acids having 10 to 24 carbon atoms, and preferably those having 10 carbons or 11 carbons with an unsaturation at omega, or ω position, i.e. at the end of the chain in relation to the acid group. For example preference is given to fatty acids with 18 carbons having an unsaturation at position δ-9 or 10 in relation to the nitrile or acid group, i.e. at position ω-9 or 8 respectively which by ethenolysis or butenolysis or other cross metathesis with an olefin will lead to ω-unsaturated acids, and to ricinoleic acid which via thermal cracking of its methyl ester gives the methyl ester of undecylenic acid.

The above-mentioned fatty acids can be isolated using any technique well known to persons skilled in the art: molecular distillation including short path distillation, crystallization, liquid-liquid extraction, complexing with urea including extraction with supercritical CO₂ and/or any combination of these techniques.

According to one particular embodiment of the method of the invention, the unsaturated fatty nitrile is obtained from a fatty acid ester, the latter advantageously being possibly selected from among the esters of the aforementioned fatty acids, in particular the methyl esters thereof. The paths to obtain a fatty nitrile from a fatty acid ester are described for example in document WO2010089512.

According to another embodiment, the unsaturated fatty nitrile is obtained from a hydroxy fatty acid such as 12HSA and 14HEA. More generally the hydroxy fatty acid can advantageously be selected from among those described in the patent application filed under number FR11.56526.

Alternatively, the unsaturated fatty nitrile is obtained from a triglyceride, the latter advantageously being possibly selected from among: a vegetable oil comprising a mixture of triglycerides of unsaturated fatty acids such has sunflower seed oil, rapeseed oil, castor oil, lesquerella, camelina, olive, soybean, palm, Sapindaceae in particular avocado, sea buckthorn, coriander, celery, dill, carrot, fennel, mango, Limnanthes Alba (meadowfoam) and the mixtures thereof; micro-algae; animal fats.

According to another embodiment, the unsaturated fatty nitrile is obtained from a vegetable wax e.g. jojoba.

The obtaining of said unsaturated fatty nitrile from an unsaturated fatty acid/ester is notably described in patent application WO201005527, in particular under the paragraphs describing the “first stage” of the method subject of this document: i.e. page 5 lines 12 to 32, page 7 lines 17 to 26, page 8 lines 1 to 9, page 10 line 29 to page 11 line 19.

According to one particular embodiment of the method of the invention, a ω-unsaturated nitrile of formula CH₂═CH—(CH₂)ₚ—CN obtained by conversion of an unsaturated fatty acid/ester in two successive steps (of indifferent order) is used: ethenolysis (cross metathesis with ethylene) and ammoniation, such as described in document WO2010055273. According to another variant of the method, hydroxylated fatty acids are used as raw material such as ricinoleic acid and lesquerolic acid which meet the general formula R₁—CH═CH—(CH₂)ₚ—COOH where R₁ is CH₃—(CH₂)₅CHOH—CH₂— and p is 7 and 9 respectively. The acid in its methyl ester form is subjected to pyrolysis leading to a ω-unsaturated ester of formula CH₂═CH—(CH₂)ₚ₊₁—COOCH₃ which is converted by ammoniation, directly or via the acid, to a ω-unsaturated nitrile. According to a further embodiment, the unsaturated fatty nitrile is produced as described in document FR11.55174 via ammoniation of a compound of fatty acid, ester or glyceride type leading to the corresponding unsaturated nitrile. According to one particular embodiment of the invention, hydrogenation of unsaturated hydroxylated fatty acids is conducted as described in the method of document FR11.56526, these fatty acids containing at least 18 carbon atoms per molecule, leading to saturated hydroxylated fatty acids, followed by dehydration thereof leading to mono-unsaturated fatty acids, with in addition either an intermediate nitrilation step of the acid function of the mono-unsaturated fatty acid leading to an unsaturated nitrile, or an intermediate nitrilation step of the acid function of the saturated hydroxylated fatty acid derived from the hydrogenation step with concomitant dehydration leading to an unsaturated fatty nitrile. Particular conditions for obtaining unsaturated fatty nitriles are described in document FR11.57542, comprising the nitrilation of a ω-unsaturated acid/ester of formula CH₂═CH(CH₂)ₙ—COOR wherein n is 7 or 8 and R is either H or an alkyl radical having 1 to 4 carbon atoms, via action of ammonia in a reactor under continuous operation in gas phase or mixed gas-liquid phase in the presence of a solid catalyst.

### 1) Hydroformylation

Hydroformylation, also called oxo method, is a synthesis path for the preparation of aldehydes from alkenes, discovered in 1938 by Otto Roelen at Ruhrchemie. The basic reaction is the following:

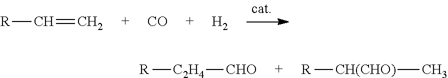

This method is widely used industrially to produce aldehydes in the C3-C19 range. Butanel is the main product synthesized by this reaction with about 75% of total production using hydroformylation as synthesis pathway. The hydroformylation step in the method of the invention uses well known methods and devices already employed in conventional hydroformylation methods. All the usual modes for the adding and mixing of reagents and components of catalyst(s) and usual separation techniques for the conventional hydroformylation reaction can therefore be applied for this step in the method of the invention. The hydroformylation step followed in the method of the invention has the advantage that it can be used directly in numerous existing devices. This would not be the case for methoxycarbonylation or hydroxycarbonylation for example.

According to the invention, the pressure conditions of the hydroformylation reaction are the following:
CO partial pressure is 40 bar or lower,
H₂ partial pressure is 40 bar or lower, and
the ratio PᵢCO/PᵢH₂ between the respective CO and H₂ partial pressures is in the range of 0.5:1 to 3:1.

In a first variant of the invention, hydroformylation is conducted under CO partial pressure in the range of 5 to 20 bar and advantageously in the range of 10 to 20 bar.

In a second more particularly advantageous embodiment, hydroformylation is conducted under CO partial pressure in the range of 5 to 40 bar and preferably in the range of 10 to 40 bar.

Advantageously, hydroformylation is conducted under H₂ partial pressure in the range of 5 to 20 bar and preferably in the range of 10 to 20 bar.

Advantageously, the PᵢCO/PᵢH₂ ratio between the respective CO and H₂ partial pressures is in the range of 1:1 to 3:1.

It is to be noted that the more the PᵢCO/PᵢH₂ ratio tends towards the value of 3:1, the more the formation of the ω-1 unsaturated isomer having the formula CH₃—CH═CH—(CH₂)ᵣ₋₁—R is promoted.

Preferably, hydroformylation is conducted at a temperature in the range of 100 to 130° C., preferably 100 to 120° C., preferably at a temperature of substantially 120° C.

Advantageously, hydroformylation is performed for a time in the range of 1 to 12 h, preferably in the range of 2 to 6 h, preferably in the range of 3 to 5 h, preferably in the order of 4 h.

Hydroformylation is preferably carried out until a conversion rate of ω-unsaturated fatty nitrile/ester/acid reactant is obtained in the range of 90 to 100%, preferably in the range of 95 to 100%, preferably in the range of 97 to 100%.

Hydroformylation is carried out in the presence of a catalyst, this catalyst comprising at least one Group VIII metal and at least one ligand, this ligand possibly being monodentate or bidentate.

Advantageously, the catalyst comprises at least one phosphine, phosphite or chelating diphosphine selected from among: PPh₃, P(OPh)₃, Dppm, Dppe, Dppb, Xantphos and/or BiPhePhos, preferably Xantphos and/or BiPhePhos, preferably BiPhePhos.

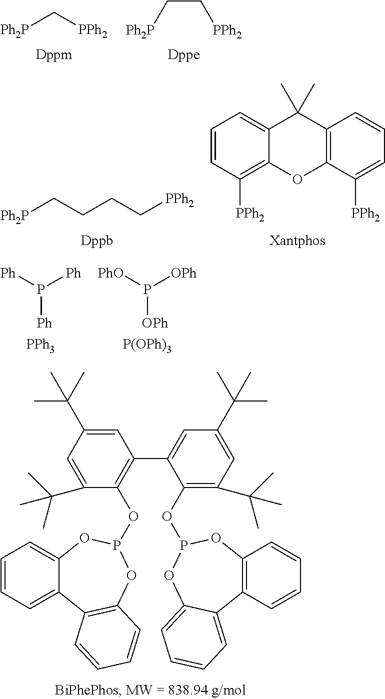

In one particularly advantageous version of the invention, the ligand of the catalyst is a bidentate ligand, which may in particular be a chelating diphosphine. The chelating diphosphine may be selected in particular from among Dppm, Dppe, Dppb, Xantphos and/or BiPhePhos. This chelating diphosphine is preferably selected from among Xantphos and/or BiPhePhos, and is more preferably BiPhePhos.

Advantageously, the metal of the catalyst is provided in the form of a precursor comprising said metal and at least one compound selected from among acetylacetonates, carbonyl compounds, cyclooctadienes, chlorine, and the mixtures thereof. Advantageously, the hydroformylation catalyst comprises rhodium, preferably provided by a precursor such as Rh(acac)(CO)₂, ruthenium, preferably provided by a precursor such as Ru₃(CO)₁₂, where acac is an acetylacetonate ligand and CO is a carbonyl ligand, and/or iridium, preferably provided by a precursor such as Ir(COD)Cl where COD is a 1,5-cyclooctadiene ligand and Cl is a chlorine ligand, preferably it comprises iridium. Advantageously hydroformylation is catalyzed by a system selected from among: Rh-Xantphos, Rh-BiPhePhos, Ir-Xantphos, Ir-BiPhePhos and the mixtures thereof.

The rhodium and iridium catalysts are preferred, they substantially improve conversion. Rhodium and iridium catalysts have better selectivity for aldehydes, cause less hydrogenation as parallel reaction and offer linear product/branched product ratios distinctly in favor of linear products.

Preferably, the hydroformylation catalyst comprises rhodium, preferably provided by a precursor such as Rh(acac)(CO)₂, ruthenium, preferably provided by a precursor such as Ru₃(CO)₁₂, where acac is an acetylacetonate ligand and CO is a carbony ligand, and/or iridium preferably provided by a precursor such as Ir(COD)Cl where COD is a 1,5-cyclooctadiene ligand and Cl is a chlorine ligand, and preferably comprises iridium. Hydroformylation is advantageously catalyzed by a system selected from among: Rh-Xantphos, Rh-BiPhePhos, Ir-Xantphos, Ir-BiPhePhos and the mixtures thereof.

Preferably the [substrate]/[metal] molar ratio is in the range of 5000 to 50 000.

This embodiment of the method of the invention is particularly advantageous in that an essentially linear hydroformylation product is obtained whilst only using a very small amount of metal, which evidently is of appreciable economic interest at industrial level.

In one particularly advantageous embodiment, the preparation method of the invention further comprises a pre-treatment step of the substrate prior to the hydroformylation step.

This pre-treatment step is intended to remove any oxidization products of the fatty nitrile/ester/acid substrate such as hydroperoxides and degradation products of these hydroperoxides, which might attack the metal of the catalyst used during the hydroformylation reaction, to the detriment of the reactivity of said catalyst.

Said pre-treatment can be performed for example by distillation of the substrate followed by purification via adsorption thereof in particular using alumina.

Preferably the [ligand]/[metal] molar ratio is in the range of 20:1 to 100:1, preferably 40:1 to 100:1.

Advantageously, hydroformylation is carried out using a sufficient amount of solvent to solubilize at least part of the catalyst (in particular one of the precursors or both precursors of the catalyst), preferably in an amount of less than 1%, preferably less than 1/1000 relative to the ω-unsaturated fatty nitrile/ester/acid reactant.

Therefore, hydroformylation can be conducted in an organic medium, e.g. in solution in toluene, but it is preferably “solvent-free”, i.e. in an amount less than 1%, preferably less than 1/1000 relative to the ω-unsaturated fatty nitrile/ester/acid reactant.

According to one particular embodiment of the method of the invention, the hydroformylation step comprises the recycling of the hydroformylation catalyst system, optionally completed by a supply of new (or “fresh”) catalyst and/or new (or “fresh”) ligand at a subsequent hydroformylation cycle.

### 2) Separation and Recovery

During the method, after halting the hydroformylation reaction, the products are evaporated from the reaction medium to recover on the one hand the isomerate and on the other hand the hydroformylation products.

Preferably, in a first fraction are recovered the isomers of the initial reactant and the initial reactant which has not reacted—and in a second fraction the nitrile (ester) aldehydes derived from the hydroformylation reaction.

Advantageously, evaporation of the reaction medium is not complete so that it is possible to recycle the catalyst and ligands back to the reaction.

When the reaction leads to an isomerate yield (containing the isomers of the initial reactant) of several percent, this mixture could be recycled back to the reaction having regard to the high cost of these raw materials. However, it is found that the mixture of isomers is much less reactive than the initial reactant which has a terminal double bond. Extensive recycling of isomerate leads to a phenomenon of build-up. In addition, these isomers continue to isomerize, the double bond continues to be shifted which in the end leads to more branched hydroformylation products (aldehydes) and diminishes the quality of the end product. The method of the invention, and in particular step 2), overcomes all these problems and, on the contrary, targets enhanced recovery of the obtained isomerate which finds numerous applications.

The hydroformylation method of the invention consumes compounds having a terminal double bond easily and rapidly. As a result, the method allows the separating of the compounds with internal double bond from the compounds with terminal double bond. Since these isomers usually have very close physicochemical properties, they are not easy to separate. Yet under the operating conditions applied in the method of the invention, the isomer with terminal double bond is converted to a linear aldehyde which means that the problem of separating internal and terminal isomers practically no longer exists. The separation of the internal isomers is thereby facilitated.

The isomerate or isomers thus isolated find applications in the field of flavorings and perfumes. For example Givaudan claimed (EP1174117) the use of said mixture of isomers in formulations. The method for producing isomerate according to the invention is also much simpler than prior art methods, as shown for example in the aforementioned Givaudan patent document in the prior art and synthesis examples 1).

The methyl esters obtained following the method of the invention an also be used for these applications. Reference can be made to the Sigma-Aldrich catalogue “Flavors and Fragrances, 2003-2004” but also to the website www.thegoodscentscompany.com which give numerous properties of these different isomers. The esters obtained can also be converted to acids, aldehydes and alcohols which in turn also have applications such as flavorings and perfumes.

Advantageously the method of the invention further comprises a step:
to separate and recover isomers from the isomerate, and/or
to convert at least one isomer of the isomerate to isomer derivative(s), in particular by conversion of one or more isomer function(s) to an acid, aldehyde, alcohol and/or amine function and/or by reaction(s) of the internal double bond of the isomers, in particular by hydrogenation, epoxidation and/or polymerization.

Advantageously, the method of the invention further comprises the valorization of the isomerate or of at least one of its isomers or the derivatives thereof, the said valorization being selected from among: use, in particular as flavoring or perfume, in a perfume composition, in particular in functional perfume products, in a composition of a cosmetic or pharmaceutical product, in the textile industry, in the metal transformation industry, as monomer in the polymer industry, in particular as monomer of an odor-free or perfumed preparation: use as lubricant, emulsifying agent, surfactant, foam reducing agent, conditioning agent, levelling agent, antistatic agent, solubilizing agent for inks, in particular printing inks, cooling fluid and/or anticorrosion agent, these products possibly being perfumed or odor-free.

A further subject of the invention is an isomerate able to be obtained with the method of the invention, characterized in that it comprises at least one fatty nitrile/ester/acid isomer with internal unsaturation, of which at least 80% of the internal isomer(s) of the isomerate are formed of the ω-1 unsaturated isomer of formula CH₃—CH═CH—(CH₂)ᵣ₋₁—R.

A further subject of the present invention is the use of an isomerate of the invention, or of at least one of the isomers thereof, or of their derivatives obtained in particular with the method of the invention, particularly used as flavoring or perfume, in a perfume composition, particularly a functional perfume, in a cosmetic or pharmaceutical composition, in the textile industry, in the metal transformation industry, as monomer in the polymer industry, in particular as monomer of an odor-free or perfumed preparation; a product used as lubricant, emulsifying agent, surfactant, foam-reducing agent, conditioning agent, levelling agent, antistatic agent, solubilizing agent for inks and in particular printing inks, cooling fluid and/or anticorrosion agent, these products possibly being odor-free or perfumed.

A further subject of the invention is a perfume composition comprising an isomerate of the invention, at least one of its isomers and/or the derivatives thereof obtained in particular using the method of the invention.

A further subject of the invention is a consumer product comprising a perfume composition of the invention.

According to one particular embodiment, the method of the invention also comprises:
2′) an oxidation step in the presence of dioxygen during which the nitrile/ester/acidaldehyde obtained at step 1) is converted to fatty nitrile/ester/acid acid of formula HOOC—(CH₂)ᵣ₊₂—R, or
2″) a reduction step, during which the nitrile/ester/acid aldehyde obtained at step 1) is converted to fatty nitrile/ester/acid alcohol of formula HO—CH₂—(CH₂)ᵣ₊₂—R or to an amino alcohol of formula HO—CH₂—(CH₂)ᵣ₊₃—NH₂ if a nitrile.

### 2′) Oxidation (or Auto-Oxidation, or Autoxidation)

After the hydroformylation step, the nitrile/ester/acid aldehyde obtained has the advantage of oxidizing very easily in contact with dioxygen. Advantageously, the oxidation step is conducted by dispersing dioxygen or a gaseous mixture containing dioxygen in the product resulting from hydroformylation. Preferably, the oxidation step is implemented without the addition of any solvent and/or without the addition of dioxygen-activating catalyst. Preferably, the oxidation step is implemented under dioxygen partial pressure ranging from 0.2 bar to 50 bar, in particular 1 bar to 20 bar, preferably 1 to 5 bar. Advantageously, the dioxygen is continuously injected into the reaction medium, preferably in the form of a stream of air or oxygen, preferably injected in excess relative to the stoichiometry of the oxidation reaction. Preferably the molar ratio of dioxygen relative to the product derived from the hydroformylation step is in the range of 3:2 to 100:2. Preferably, oxidation is conducted at a temperature in the range of 0° C. to 100° C., preferably 20° C. to 100° C., more preferably 30° C. to 90° C., further preferably 40° C. to 80° C., optionally with two consecutive increasing temperature holds.

Advantageously the method of the invention further comprises:
3′) a reduction step during which the nitrile acid obtained at step 2) is converted to an ω-amino acid of formula HOOC—(CH₂)ᵣ₊₃—NH₂ for a nitrile acid; or
3″) a hydrolysis step during which the ester acid obtained at step 2) is converted to a diacid of formula HOOC—(CH₂)ᵣ₊₂—COOH for an ester acid.

According to one particular embodiment, the method of the invention further comprises a step to synthesize a polymer, polyamide in particular, by polymerization using the ω-amino acid or diacid obtained at step 3′); or a polyester by polymerization using the ester alcohol obtained at step 2″).

### 3′) Reduction or Hydrogenation of the Nitrile Function to an Amine

The synthesis step of fatty ω-amino esters or ω-amino acids from the fatty ester nitriles or acid nitriles respectively entails conventional reduction or hydrogenation. The reduction of the nitrile function to a primary amine is well known to persons skilled in the art. Hydrogenation is performed for example in the presence of precious metals (Pt, Pd, Rh, Ru . . . ) at a temperature between 20 and 100° C. under pressure of 1 to 100 bar, and preferably 1 to 50 bar. It can also be performed in the presence of catalysts containing iron, nickel or cobalt which can withstand more severe conditions with temperatures in the order of 150° C. and high pressures of several tens of bars. To promote the formation of the primary amine, ammonia partial pressure is preferably applied. Advantageously the reduction step of the fatty acid nitriles to ω-amino fatty acid uses hydrogenation with any conventional catalyst, and preferably Raney nickel and cobalt catalysts, in particular Raney nickel whether or not deposited on a substrate such as silica.

### Synthesis of Polyamides

The polymers able to be produced from the fatty nitrile/ester/acid aldehydes of the present invention are specialty products, e.g. technical polyamides with respect to polyamides, i.e. high-performing even very high-performing polyamides produced from precursors or monomers having at least 8 carbon atoms, preferably at least 10 carbon atoms; as opposed to so-called “commodity” polyamides, such as “nylon-6”, for which the marketing quantities (volumes) are very much higher and the costs much lower than for technical polyamides.

According to one particular embodiment, the method of the invention is a method to synthesize a ω-amino acid compound of formula:

HOOC—(CH₂)ᵣ₊₂—CH₂NH₂,

from a mono-unsaturated fatty nitrile compound of formula:

CH₂═CH—(CH₂)ᵣ—CN

comprising the following steps:
hydroformylation of the unsaturated nitrile compound to obtain a nitrile aldehyde compound of formula HOC—(CH₂)ᵣ₊₂—CN, then
oxidation of the nitrile aldehyde compound to obtain the corresponding nitrile acid compound of formula HOOC—(CH₂)ᵣ₊₂—CN, and
reducing the nitrile-acid compound to ω-amino acid of formula

HOOC—(CH₂)ᵣ₊₂—CH₂NH₂.

According to one advantageous embodiment, the method of the invention also comprises a step to synthesize a polyamide by polymerization using the ω-amino acid obtained at step 3).

Starting from castor oil for example, methanolysis is carried out to obtain methyl ricinoleate of formula:

CH₃—(CH₂)₅—CHOH—CH₂—CH═CH—(CH₂)₇—COOCH₃

after which thermal cracking is performed to obtain methyl undecylenate:

CH₂═CH—(CH₂)₈—COOCH₃

which can be hydrolyzed to undecylenic acid:

CH₂═CH—(CH₂)₈—COOH

followed by a nitrilation step to obtain the undecenenitrile:

CH₂═CH—(CH₂)₈—CN

Alternatively, the conversion of the methyl ester of undecylenic acid to a nitrile is performed directly.

The undecenenitrile thus obtained is used in the method of the invention at the following steps:
1) hydroformylation of the nitrile in the presence of CO and H₂, to obtain a nitrile aldehyde having 12 carbons:

HOC—(CH₂)₁₀—CN
2) autoxidation of the aldehyde to acid, to obtain:

HOOC—(CH₂)₁₀—CN
3) reduction of the nitrile, to obtain the C12 amino acid:

HOOC—(CH₂)₁₀—CH₂—NH₂

which, via polymerization, allows the production of polyamide-12 from a renewable source.

Starting from an oil high in oleic acid (cis-9-octadecenoic acid) of formula:

CH₃—(CH₂)₇—CH═CH—(CH₂)₇—COOR,
R being a glyceric radical meeting formula —CH₂—CHOX—CH₂OY, X and Y each independently being H, another fatty chain of the triglyceride (oil) or oleic radicals,
   it is possible to proceed as follows:
ethenolysis (cross metathesis with ethylene or other alpha olefin), to obtain at least:

CH₃—(CH₂)₇—CH═CH₂+CH₂═CH—(CH₂)₇—COOR
methanolysis and separation of the fatty acids to isolate the methyl decenoate:

CH₂═CH—(CH₂)₇—COOCH₃
hydrolysis of the ester to acid CH₂═CH—(CH₂)₇—COOH,
nitrilation of the acid to 9-decenenitrile CH₂═CH—(CH₂)₇—CN

Thereafter the 9-decenenitrile is subjected to the following steps according to the method of the invention:
hydroformylation of the 9-decenenitrile to C11 nitrile aldehyde: HOC—(CH₂)₉—CN
autoxidation to form the C11 nitrile acid: HOOC—(CH₂)₉—CN
reduction to form 11-aminoundecanoic acid HOOC—(CH₂)₉—CH₂—NH₂.

Via polymerization of 11-aminoundecanoic acid, polyamide-11 is produced.

Alternatively the oleic acid can be converted to oleic nitrile, followed by ethenolysis (or other cross metathesis with an alpha-olefin) to obtain the nitrile containing 10 carbon atoms.

### EXAMPLES

Unless otherwise indicated, all percentages are percentages in number of moles.

### 1. Materials

Rh(acac)(CO)₂ (marketed by STREM) was used as precursor for the hydroformylation catalyst. The phosphines (marketed by STREM) were used such as received or synthesized.

2. Substrate: 10-undecenenitrile (or 1,10-undecenitrile)

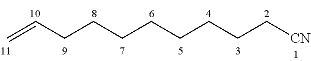

### 3. Hydroformylation Reaction

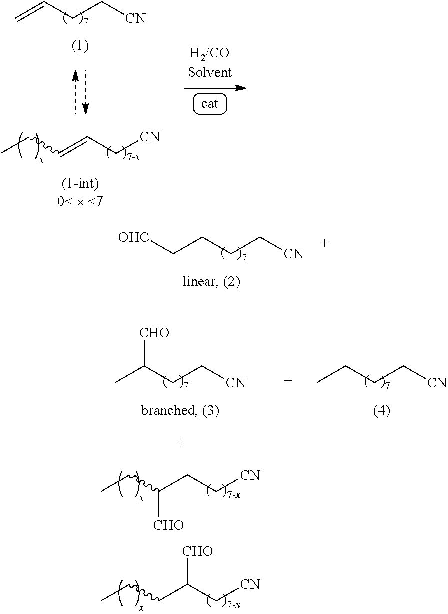

The isomerate (1-int) contains a mixture of isomers with internal unsaturation (ω-x : ω-1, ω-2, ω-3 . . . ) of the substrate (1) with terminal unsaturation (ω-unsaturated):

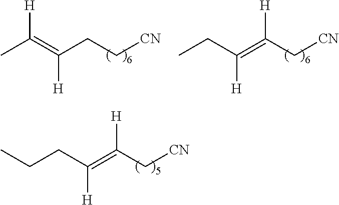

For simplification solely the isomers of trans type are illustrated in the above schemes. Evidently the isomers of cis type are also produced.

### General Procedure:

The hydroformylation reactions are carried out in a 100 mL stainless steel autoclaves. Under typical conditions, a solution in toluene (0.5 to 5 mL) of metal precursor (0.001 to 0.0001 mmol), phosphine (0.002 to 0.02 mmol) and substrate (5 to 25 mmol) is mixed in a Schlenk tube under argon inert atmosphere to form a homogeneous solution. After agitation at ambient temperature for 1 hour, this solution is cannulated into the autoclave previously provided with an inert atmosphere. The reactor is sealed, purged several times with a CO/H₂ mixture (1:1), pressurized at 20 bar of this CO/H₂ mixture at ambient temperature and heated to the desired temperature using a hot water bath or oil bath. During the reaction, the pressure is held constant and several samples are taken to monitor conversion. After a suitable reaction time, the autoclave is returned to ambient temperature and then atmospheric pressure. The mixture is collected and analyzed by NMR.

### Example 1

### Hydroformylation of 10-undecenenitrile (Rh-biphephos with S/Rh=20,000 and L/Rh=20)

A solution of Rh(acac)(CO)₂ in toluene (0.65 mg, 0.00025 mmol), Biphephos (4 mg, 0.005 mmol) and undecenitrile (826 mg, 5.0 mmol) was prepared in a Schlenk tube under argon inert atmosphere to form a homogeneous solution left under agitation at ambient temperature for 1 h. The Biphephos/rhodium molar ratio was 20:1 and substrate/rhodium molar ratio 20,000:1. The solution was cannulated into a 100 ml autoclave previously provided with an inert atmosphere. The reactor was sealed, purged several times with a CO/H₂ gas mixture (1:1), pressurized at 20 bar CO/H₂ (1:1) at ambient temperature and heated to 120° C. After 5 h, 100% undecenitrile was consumed. After 5 h, the medium was returned to ambient temperature and atmospheric pressure. The mixture was collected and analyzed by NMR. Analysis showed that the reaction was complete and that there remained a proportion of internal olefin of 19%, a proportion of hydrogenation product (4) of 5%, and that 86% of the products formed corresponded to branched aldehydes (1%) and linear aldehydes (99%).

For subsequent valorization of the formed sub-products, the internal alkenes were separated from the mixture by Kugelrhor distillation (135° C., 1 mbar). ¹³C NMR analysis showed that 80% of these internal alkenes were 9-undecenenitrile whereas 20% represented 8-undecenitrile.

### Example 2

### Hydroformylation of 10-undecenenitrile (Rh-biphephos with S/Rh=20,000 and L/Rh=100, 5 h)

A solution of Rh(acac)(CO)₂ in toluene (0.65 mg, 0.00025 mmol), Biphephos (20 mg, 0.025 mmol) and undecenitrile (826 mg, 5.0 mmol) was prepared in a Schlenk tube under argon inert atmosphere to form a homogeneous solution which was left under agitation at ambient temperature for 1 h. The Biphephos/rhodium molar ratio was 100:1 and the molar ratio of substrate/rhodium 20,000:1. The solution was cannulated into a 100 mL autoclave previously provided with an inert atmosphere. The reactor was sealed, purged several times with CO/H₂ gas mixture (1:1), then pressurized at 20 bar CO/H₂ (1:1) at ambient temperature and heated to 120° C. After 4 h, the medium was returned to ambient temperature and atmospheric pressure. The mixture was collected and analyzed by NMR. Analysis showed that the reaction was complete and that there remained a 21% proportion of internal olefin, 5% proportion of hydrogenation product and that 84% of the formed products corresponded to branched aldehydes (1%) and linear aldehydes (99%).

The internal alkenes were also separated from the mixture by Kugelrhor distillation (135° C., 1 mbar). ¹³C NMR analysis showed that 95% of these alkenes were 9-undecenitrile whereas 5% represented 8-undecenitrile.

### Example 3

### Hydroformylation of 10-undecenenitrile (Rh-biphephos with S/Rh=50,000 and L/Rh=20; solvent-free)

A solution of Rh(acac)(CO)₂ (0.13 mg, 0.0005 mmol) in toluene (0.5 ml), Biphephos (8 mg, 0.01 mmol) and undecenitrile (4.12 g, 25.0 mmol) was prepared in a Schlenk tube under argon inert atmosphere to form a homogeneous solution which was left under agitation at ambient temperature for 1 h. The Biphephos/rhodium molar ratio was 20:1 and substrate/rhodium molar ratio 50,000:1. The solution was cannulated into a 100 mL autoclave previously provided with an inert atmosphere. The reactor was sealed, purged several times with CO/H₂ gas mixture (1:1), then pressurized at 20 bar CO/H₂ (1:1) at ambient temperature and heated to 120° C. After 4 h, 100% of the undecenitrile had been consumed. The reaction was left to continue for 48 h for maximum internal olefin consumption. The mixture was collected and analyzed by NMR after 48 h. Analysis showed that the reaction was complete and that there remained a 6% proportion of internal olefin, 7% hydrogenation product and 87% of the formed products corresponded to branched aldehydes (1%) and linear aldehydes (99%).

### Example 4

### Kinetics of the Hydroformylation Reaction, Solvent-Free and in Solution (Rh-biphephos with S/Rh=20,000 and L/Rh=20;)

TABLE 1^{[a]}

| | [1]₀/ | biphephos | [1]₀ | Time | 1 | 1-int w | w-1 | w-2 | 2 + 3 | 4 | Conv. 1 | HF | |
|---|---|---|---|---|---|---|---|---|---|---|---|---|---|
| Input | [Rh] | [eq vs. Rh] | [M] | [h] | [%]^{[b]} | [%]^{[b]} | [%]^{[b]} | [%]^{[b]} | [%]^{[b]} | [%]^{[b]} | [%]^{[c]} | [%]^{[d]} | 2/3 |
| 1 | 20000 | 20 | 1M | 2 | 30 | 17 | 82 | 18 | 49 | 4 | 68 | 75 | 99/1 |
| | | | | 3 | 6 | 22 | 71 | 29 | 68 | 4 | 94 | 76 | 99/1 |
| | | | | 4 | 0 | 20 | 74 | 26 | 75 | 5 | 100 | 79 | 99/1 |
| | | | | 5 | 0 | 19 | 75 | 25 | 76 | 5 | 100 | 80 | 99/1 |
| 2^{[e]} | 20000 | 20 | solvent-free^{[e]} | 2 | 25 | 15 | 84 | 16 | 56 | 4 | 74 | 80 | 99/1 |
| | | | | 3 | 4 | 21 | 78 | 22 | 69 | 6 | 96 | 76 | 99/1 |
| | | | | 4 | 0 | 20 | 80 | 20 | 74 | 6 | 100 | 78 | 99/1 |
| | | | | 5 | 0 | 19 | 81 | 19 | 75 | 6 | 100 | 79 | 99/1 |

^{[a]}Reaction conditions unless otherwise specified: 1/1-int (95:5 mixture) = 5 mmol, toluene (5 ml), P = 20 bar CO/H₂ (1:1).
^{[b]}Distribution (% mol) of remainder 1, internal alkenes 1-int (residual or formed during the reaction), aldehydes 2 and 3, and hydrogenation product 4, such as determined by NMR and GLC.
^{[c]}Conversion of 1.
^{[d]}Selectivity for hydroformylation products (2 + 3).
^{[e]}A minimum amount of toluene (0.5 mL) was used to add the catalyst precursors. It is considered to be solvent-free hydroformylation.

### Example 5

### Hydroformylation of 1,10-undecenenitrile (Ir-biphephos with S/Ir=20,000 and L/Ir=20) (Input 2)

A solution of [Ir(COD)(Cl)]₂ (0.83 mg, 0.00025 mmol) in toluene (5 mL), Biphephos (4 mg, 0.005 mmol) and undecenitrile (5.0 mmol) was prepared in a Schlenk tube under argon inert atmosphere to form a homogeneous solution then left under agitation at ambient temperature for 1 h. The Biphephos/iridium molar ratio was 20:1 and substrate/iridium molar ratio 20,000:1. The solution was cannulated into a 100 mL autoclave previously provided with an inert atmosphere. The reactor was sealed, purged several times with CO/H₂ gas mixture (1:1), then pressurized at 20 bar CO/H₂ (1:1) at ambient temperature and heated to 120° C. After 18 h, 100% of the undecenenitrile had been consumed. The mixture was collected and analyzed by NMR after 18 h. Analysis showed that the reaction was complete and that there remained a 22% proportion of internal olefin (of which 85% 9-undecenenitrile), 5% hydrogenation product while 73% of the formed product corresponded to branched aldehydes (<1%) and linear aldehydes (>99%).

The protocol of Example 5 above was reproduced with another ligand (Xantphos, Input 1) or another solvent (THF or NMP, Inputs 3 and 4 respectively).

All results are given in Table 2 below:

TABLE 2^{[a]}

| | | | 1 | 1-int | 9-undec | 8-undec | 2 + 3 | 4 | Conv. 1 | HF | |
|---|---|---|---|---|---|---|---|---|---|---|---|
| Input | Ligand | Solvent | [%]^{[b]} | [%]^{[b]} | [%]^{[b]} | [%]^{[b]} | [%]^{[b]} | [%]^{[b]} | [%]^{[c]} | [%]^{[d]} | 2/3 |
| 1 | Xantphos | Toluene | 82 | 7 | 95 | 5 | 9 | 2 | 14 | 69 | 98/2 |
| 2 | Biphephos | Toluene | 0 | 22 | 84 | 16 | 73 | 5 | 100 | 77 | >99/1 |
| 3 | Biphephos | THF | 0 | 26 | 88 | 12 | 69 | 5 | 100 | 73 | >99/1 |
| 4 | Biphephos | NMP | 25 | 18 | 95 | 5 | 52 | 5 | 74 | 74 | >99/1 |

^{[a]}Reaction conditions unless otherwise specified: 1/1-int (95:5 mixture) = 5.0 mmol, [1/1-int]₀/[Ir] = 20,000, [ligand]/[Ir] = 20, P = 20 bar CO/H₂ (1:1), 5 mL of solvent.
^{[b]}Distribution (% mol) of remainder 1, internal alkenes 1-int (residual or formed during the reaction), aldehydes 2 and 3, and hydrogenation product 4, such as determined by NMR and GLC analysis.
^{[c]}Conversion of 1.
^{[d]}Selectivity for hydroformylation products (2 + 3).

### Example 6

### Hydroformylation of methyl-10-undecenoate (Rh-biphephos with S/Rh=50,000 and L/Rh=20; solvent-free)

A solution of Rh(acac)(CO)₂ (0.13 mg, 0.0005 mmol) in toluene (0.5 ml), Biphephos (8 mg, 0.01 mmol) and methyl-10-undecenoate (25.0 mmol) was prepared in a Schlenk tube under argon inert atmosphere to form a homogeneous solution then left under agitation at ambient temperature for 1 h. The Biphephos/rhodium molar ratio was 20:1 and substrate/rhodium molar ratio 50,000:1. The solution was cannulated into a 100 mL autoclave previously provided with an inert atmosphere. The reactor was sealed, purged several times with CO/H₂ gas mixture(1:1), then pressurized at 20 bar CO/H₂ (1:1) at ambient temperature and heated to 120° C. After 5 h, 100% of the undecenoate has been consumed. The reaction was left to continue for 48 h for maximum internal olefin consumption. The mixture was collected and analyzed by NMR after 48 h. Analysis showed that the reaction was complete and that there remained a 6% proportion of internal olefin, 9% hydrogenation product while 85% of the formed products corresponded to branched aldehydes (1%) and linear aldehydes (99%).

### Example 7

### Oxidation of Hydroformylation Products

The aldehydes resulting from hydroformylation of the previously distilled undecenitrile (5.4 g, 30 mmol) were dissolved in ether (10 mL) and left under agitation in air for 48 h. A white crystalline solid was gradually formed and collected by filtration; it corresponded to the corresponding carboxylic acid.

### Example 8

### Hydrogenation of the Nitrile Acid

A solution of Ni/Raney (40 mg) in water/ethanol mixture (2 ml/2 mL), ammonia (3 mmol, 1M in methanol) and nitrile acid (360 mg, 2 mmol) was prepared in a Schlenk tube at ambient temperature. The solution was cannulated into a 100 mL autoclave previously provided with an inert atmosphere. The reactor was sealed, purged several times with H₂ gas then pressured at 40 bar H₂ at ambient temperature and heated to 130° C. After 12 h, 100% of the nitrile acid had been hydrogenated. The reactor was depressurized at ambient temperature and 5 mL of acetic acid were added. The catalyst was extracted by filtration and the filtrate evaporated. A white precipitate was obtained after washing in ether (15 mL). NMR and IR analysis showed that the zwitterionic product had been obtained.

### Example 9

### Comparison Between the Effect of 2 Different Ligands

Conditions: S/Rh=20,000, 20 bar CO/H₂ (1:1), 120° C., 5 h, Toluene (5 ml), [1+1-int]0=5 mmol.

TABLE 3

| | | | | | | | | | Hydrogenated |
|---|---|---|---|---|---|---|---|---|---|
| | | Ligand | Conv. | 1-int | % 9- | % 8- | Sel. (%)[b] | | product |
| Input | Ligand | [equiv]0 | (%) | total | undecen | undecen | 2 | 3 | 4 |
| 1 | Xantphos | 100 | 42 | 8 | 95 | 5 | 98 | 2 | 4 |
| 2 | Biphephos | 20 | 100 | 19 | 79 | 21 | 99 | 1 | 5 |
| 3 | Biphephos | 100 | 100 | 21 | 95 | 5 | 99 | 1 | 5 |

Observations:

A large excess of ligand (100 Eq for Inputs 1 and 3 in Table 3) in relation to the undecenenitrile substrate:
does not lead to a global reduction in isomerization products (19% vs. 21% -Input 2 vs Input 3);
on the other hand, it has a positive effect on the distribution of internal olefins by increasing the content of ω-1 unsaturated isomers compared to ω-2 unsaturated ones. The isomerization reaction was slowed beyond the ω-1 position due to the presence of ligand excess. If too much ligand, the latter ends up competing with the reactants for access to the metal.

The following examples show that isomerization is limited with larger ligand quantity.

Experiments were conducted to evaluate the importance of excess ligand in the hydroformylation-isomerization reaction of 1, in particular on the distribution of internal isomers 1-int.

The results are grouped together in Table 4.

With Rh(acac)CO₂, [Ir(cod)Cl]₂ and Ir(cod)(acac) as catalysts, a notable positive effect was observed on selectivity for 9-undecenitrile by increasing the [biphephos]/[metal] ratio from 20 to 100 (see Table 4, Inputs 1-3, 4-6 and 7-8).

TABLE 4^{[a]}

| | | biphephos | 1 | 1-int | 9-/8- | 2 + 3 | | 4 | Conv. 1 | HF |
|---|---|---|---|---|---|---|---|---|---|---|
| Input | Catalyst | [equiv]₀ | [%]^{[b]} | [%]^{[b]} | undec | [%]^{[b]} | 2/3 | [%]^{[b]} | [%]^{[c]} | [%]^{[d]} |
| 1^{[e]} | Rh(acac)(CO)₂ | 20 | 1 | 17 | 79/21 | 77 | 99/1 | 5 | 99 | 82 |
| 2^{[e]} | Rh (acac) (CO)₂ | 50 | 1 | 17 | 84/16 | 77 | 99/1 | 5 | 99 | 82 |
| 3^{[e]} | Rh (acac) (CO)₂ | 100 | 1 | 19 | 90/10 | 76 | 99/1 | 4 | 99 | 81 |
| 4 | [Ir(cod)Cl]₂ | 20 | 0 | 22 | 86/14 | 73 | 99/1 | 5 | 100 | 77 |
| 5 | [Ir(cod)Cl]₂ | 50 | 0 | 23 | 93/7 | 71 | 99/1 | 6 | 100 | 75 |
| 6 | [Ir(cod)Cl]₂ | 100 | 18 | 16 | 100/0 | 60 | 99/1 | 6 | 81 | 78 |
| 7 | Ir(cod)(acac) | 20 | 3 | 21 | 92/8 | 64 | 99/1 | 12 | 97 | 70 |
| 8 | Ir(cod)(acac) | 100 | 36 | 13 | 95/5 | 43 | 99/1 | 8 | 62 | 73 |

^{[a]}Reaction conditions unless otherwise specified: 1/1-int (95:5 mixture) = 5.0 mmol, [1/1-int]₀/[M] = 20,000, [ligand]/[Ir] = 20, P = 20 bar CO/H₂ (1:1), 5 mL toluene, 120° C., reaction time: 5 h (Rh) and 20 h (Ir).
^{[b]}Distribution (% mol) of remainder 1, internal alkenes 1-int, aldehydes 2 and 3, and hydrogenation product 4, such as determined by NMR and GLC analysis.
^{[c]}Conversion of 1.
^{[d]}Selectivity for hydroformylation products (2 + 3)

### Example 10

### Study on the Effect of the CO/H₂ Ratio

Conditions: S/Rh=20,000, L/Rh=20, L: Biphephos, 120° C., 5 h, Toluene (5 ml), [1+1-int]0=5 mmol.

TABLE 5

| | | | | | | | | | Sel. (%)[b] | | | |
|---|---|---|---|---|---|---|---|---|---|---|---|---|
| | Total P | CO | H₂ | | Conv. | 1-int | % 9- | % <8- | | | ald. | Hydrogenated |
| Input | (bar) | (bar) | (bar) | CO/H₂ | (%) | alkene[c] | undecen | undecen | 2 | 3 | Int. | product 4 |
| 1 | 20 | 10 | 10 | 1/1 | 100 | 19 | 79 | 21 | 99 | 1 | — | 5 |
| 2 | 20 | 5 | 15 | 1/3 | 100 | 18 | 74 | 26 | 99 | 1 | — | 7 |
| 3 | 20 | 15 | 5 | 3/1 | 100 | 28 | 77 | 23 | 97 | 3 | — | 25 |

Observations: The CO/H₂ ratio at 20 bar and after 5 h:
influences the distribution of internal olefins: the content of ω-1 unsaturated isomers increases compared to ω-2 unsaturated isomers content with the CO/H₂ ratio;
more especially influences selectivity: the more the CO/H₂ ratio decreases, the more selectivity increases (fewer hydrogenation products are formed).

### Example 11

### Comparison of Different [Ligand]/[Metal] Ratios:

TABLE 6

| | | | | | | | | Sel. (%)[b] | | | |
|---|---|---|---|---|---|---|---|---|---|---|---|
| | | | Time | Conv. | 1-int | % 9- | % 8- | | | sub- | Hydrogenated |
| Input | Substrate | L/Rh | (h) | (%)[c] | total | undecen | undecen | n-2 | iso-3 | pdt | product |
| 1* | Methyl 10- | 20 | 5 | 100 | 12 | 63 | 37 | 99 | 1 | — | 8 |
| | undecenoate | | | | | | | | | | |
| 1** | Methyl 10- | 100 | 5 | 100 | 12 | 82 | 18 | 99 | 1 | — | 8 |
| | undecenoate | | | | | | | | | | |
| 2* | 10- | 20 | 5 | 100 | 16 | 79 | 21 | 99 | 1 | — | 5 |
| | undecenenitrile | | | | | | | | | | |
| 2** | 10- | 100 | 5 | 100 | 20 | 95 | 5 | 99 | 1 | — | 6 |
| | undecenenitrile | | | | | | | | | | |

Observations:

The more the ligand/metal ratio increases, the more the content of ω-1 unsaturated isomers increases compared to the content of ω-2 unsaturated isomers.

### Example 12

### Effect of Total CO/H₂ Pressure on Hydroformylation-Isomerization of 1/1-int Catalyzed by Rh- and Ir-biphephos

The impact of pressure was investigated under optimized conditions for Ir- and Rh-biphephos catalysts. The results obtained are grouped together in Table 7 below.

Irrespective of the pressure applied (20 or 80 bar), the activity and selectivity for aldehyde 2 are not affected (Table 7, Inputs 1-4 and 5-8).

However, the percentage of internal olefins as formed decreases with pressure increase (Table 7, Inputs1-4 and 5-8).

For both types of catalyst, a higher pressure generates better selectivity to the benefit of hydroformylation and better control over the distribution of internal isomers to the benefit of 9-undecenenitrile (in ranges of 86-95% with Rh and 90-97% with Ir).

TABLE 7^{[a]}

| | | H₂/CO | 1 | 1-int | 9-/8- | 2 + 3 | | 4 | Conv. 1 | HF |
|---|---|---|---|---|---|---|---|---|---|---|
| Input | Catalyst | [bar] | [%]^{[b]} | [%]^{[b]} | undec | [%]^{[b]} | 2/3 | [%]^{[b]} | [%]^{[c]} | [%]^{[d]} |
| 1 | Rh(acac)(CO)₂ | 10 | 0 | 37 | 86/14 | 58 | 99/1 | 5 | 100 | 60 |
| 2^{[e]} | Rh(acac) (CO)₂ | 20 | 0 | 21 | 89/11 | 75 | 99/1 | 4 | 100 | 79 |
| 3 | Rh(acac)(CO)₂ | 40 | 0 | 18 | 93/7 | 78 | 99/1 | 4 | 100 | 82 |
| 4 | Rh(acac)(CO)₂ | 80 | 0 | 15 | 95/5 | 82 | 99/1 | 3 | 100 | 86 |
| 5 | Ir(cod)(acac) | 10 | 2 | 35 | 90/10 | 58 | 99/1 | 5 | 98 | 63 |
| 6 | Ir(cod)(acac) | 20 | 1 | 20 | 92/8 | 75 | 99/1 | 4 | 99 | 80 |
| 7 | Ir(cod)(acac) | 40 | 2 | 21 | 95/5 | 73 | 99/1 | 4 | 97 | 79 |
| 8 | Ir(cod)(acac) | 80 | 3 | 17 | 97/3 | 77 | 99/1 | 3 | 97 | 84 |

^{[a]}Reaction conditions unless otherwise specified: 1/1-int (95:5 mixture) = 5.0 mmol, [1/1-int]₀/[Ir] = 20,000 and [1/1-int]₀/[Rh] = 50,000, [ligand]₀/[M] = 20, solvent (5 mL): toluene (Rh) or acetonitrile (Ir), 120° C., reaction time: 5 h (Rh) and 20 h (Ir,), CO/H₂ pressure (1:1).
^{[b]}Distribution (% mol) of remainder 1, internal alkenes 1-int, aldehydes 2 and 3, and hydrogenation product 4, such as determined by NMR and GLC analysis.
^{[c]}Conversion of 1.
^{[d]}Selectivity for hydroformylation products (2 + 3)

### Example 13

### Effect of CO/H₂ Ratio at 40 Bar on Hydroformylation-Isomerization of 1/1-int Catalyzed by Rh- and Ir-biphephos

According to the results of Table 8 below, it is observed that an increase in CO partial pressure improves chemoselectivity and hence yields of aldehydes, and allows reduced isomerization (less shifting of the double bond along the chain).

In addition, fewer hydrogenation products 4 are formed. This finding appears to show that at higher total pressure, there is promoting of the hydroformylation reaction over hydrogenation.

Concomitantly, better control over the distribution of internal isomers is achieved.

These observations confirm those already made in Example 10, according to Table 5.

TABLE 8^{[a]}

| | | CO/H₂ | 1 | 1-int | 9-/8- | 2 + 3 | | 4 | Conv. 1 | HF |
|---|---|---|---|---|---|---|---|---|---|---|
| Input | Catalyst | at 40 bar | [%]^{[b]} | [%]^{[b]} | undec | [%]^{[b]} | 2/3 | [%]^{[b]} | [%]^{[c]} | [%]^{[d]} |
| 1 | Rh(acac)(CO)₂ | 1/1 | 0 | 18 | 93/7 | 78 | 99/1 | 4 | 100 | 82 |
| 2 | Rh(acac)(CO)₂ | 1/3 | 0 | 20 | 85/15 | 71 | 96/4 | 9 | 100 | 74 |
| 3 | Rh(acac)(CO)₂ | 3/1 | 0 | 16 | 94/6 | 81 | 99/1 | 3 | 100 | 85 |
| 4 | Ir(cod)(acac) | 1/1 | 2 | 21 | 95/5 | 73 | 99/1 | 4 | 97 | 79 |
| 5 | Ir(cod)(acac) | 1/3 | 18 | 22 | 90/10 | 54 | 97/3 | 6 | 81 | 70 |
| 6 | Ir(cod)(acac) | 3/1 | 52 | 9 | 96/4 | 38 | 99/1 | 1 | 45 | 88 |

^{[a]}Reaction conditions unless otherwise specified: 1/1-int (95:5 mixture) = 5.0 mmol, [1/1-int]₀/[M] = 20,000, [biphephos]₀/[M] = 20, solvent (5 mL): toluene (Rh) or acetonitrile (Ir), CO/H₂ total pressure of 40 bar, 120° C., reaction time: 5 h (Rh) and 20 h (Ir).
^{[b]}Distribution (% mol) of remainder 1, internal alkenes 1-int, aldehydes 2 and 3, and hydrogenation product 4, such as determined by NMR and GLC analysis.
^{[c]}Conversion of 1.
^{[d]}Selectivity for hydroformylation products (2 + 3)

### Example 14

### Bulk, Solvent-Free Hydroformylation and Isomerization Reaction

TABLE 9^{[a]}

| | | | | | | 9-/8- | | | | | | |
|---|---|---|---|---|---|---|---|---|---|---|---|---|
| | Catalyst | | Time | 1 | 1-int | undec | Trans/ | 2 + 3 | | 4 | Conv. 1 | HF |
| Input | [M] | [1]₀/[Rh] | [h] | [%]^{[b]} | [%]^{[b]} | ratio | Cis | [%]^{[b]} | 2/3 | [%]^{[b]} | [%]^{[c]} | [%]^{[d]} |
| 1 | Rh(acac)(CO)₂ | 20000 | 5 | 1 | 20 | 84/16 | 65/35 | 75 | 99/1 | 4 | 99 | 80 |
| 2 | Rh(acac)(CO)₂ | 50000 | 5 | 9 | 21 | 89/11 | 62/38 | 65 | 99/1 | 5 | 91 | 76 |
| 3 | Ir(cod)(acac) | 20000 | 20 | 8 | 16 | 94/6 | 60/40 | 72 | 99/1 | 4 | 92 | 83 |
| 4^{[e]} | Ir(cod)(acac) | 20000 | 20 | 18 | 14 | 95/5 | 58/42 | 63 | 99/1 | 4 | 81 | 83 |

^{[a]}Reaction conditions unless otherwise specified: 1/1-int (95:5 mixture) = 25.0 mmol, [biphephos]₀/[M] = 20, CO/H₂ pressure(1:1) = 40 bar, 120° C., minimum content (0.5 mL) of toluene (Rh) or acetonitrile (Ir) was used to add catalyst precursors.
^{[b]}Distribution (% mol) of the remainder 1, internal alkenes 1-int, aldehydes 2 and 3, and hydrogenation product 4, such as determined by NMR and GLC analysis.
^{[c]}Conversion of 1.
^{[d]}Selectivity for hydroformylation products (2 + 3)
^{[e]}P = 80 bar CO/H₂ (1:1).

### Example 15

### Hydroformylation Reaction of 1/1-int in the Presence of Rh-biphephos

TABLE 10^{[a]}

| | | | | | | 9/8- | | | | | | |
|---|---|---|---|---|---|---|---|---|---|---|---|---|
| | | T | H₂/CO | 1 | 1-int | undec | Trans/ | 2 + 3 | | 4 | Conv. 1 | HF |
| Input | Catalyst | [° C.] | [bar] | [%]^{[b]} | [%]^{[b]} | ratio | Cis | [%]^{[b]} | 2/3 | [%]^{[b]} | [%]^{[c]} | [%]^{[d]} |
| 1 | Rh(acac)(CO)₂ | 120 | 20 | 0 | 21 | 89/11 | 68/32 | 75 | 99/1 | 4 | 100 | 79 |
| 2^{[e]} | Rh(acac)(CO)₂ | 80 | 20 | 0 | 17 | 90/10 | 65/35 | 78 | 99/1 | 5 | 100 | 82 |
| 3 | Rh(acac)(CO)₂ | 120 | 40 | 0 | 18 | 93/7 | 62/38 | 78 | 99/1 | 4 | 100 | 82 |
| 4 | Rh(acac)(CO)₂ | 120 | 80 | 0 | 15 | 95/5 | 58/42 | 82 | 99/1 | 3 | 100 | 86 |

^{[a]}Reaction conditions unless otherwise specified: 1/1-int (95:5 mixture) = 5.0 mmol, [substrate]₀/[Rh] = 20,000, [biphephos]₀/[M] = 20, toluene (5 mL), 5 h, CO/H₂ pressure (1:1).
^{[b]}Distribution (% mol) of remainder 1, internal alkenes 1-int, aldehydes 2 and 3, and hydrogenation product 4, such as determined by NMR and GLC analysis.
^{[c]}Conversion of 1.
^{[d]}Selectivity for hydroformylation products (2 + 3)
^{[e]}Reaction time = 20 h.

It is observed that total pressure forms one means to control isomerization, by acting on shifting of the double bond, and the ratio between isomers with cis and trans internal unsaturation.

### Example 16

### Effect of CO/H₂ Ratio on Hydroformylation-Isomerization of 1/1-int Catalyzed by Rh- and Ir-biphephos

The catalysts Ir- and Rh-biphephos also allow the hydroformylation and isomerization of unsaturated fatty esters such as methyl-10-undecenoate (R═COOCH₃).

Results under the conditions used for conversion of the unsaturated nitrile are grouped together in Table 11 below.

Good chemoselectivity and excellent regioselectivity for the formation of linear aldehydes are obtained.

TABLE 11^{[a]}

| | | | | | | | | Conv. | |
|---|---|---|---|---|---|---|---|---|---|
| | | Subs | int | 9-/8- | ald | Sel | H₂ | Subs | HF |
| Input | Catalyst | [%]^{[b]} | [%]^{[b]} | undec | [%]^{[b]} | ald | [%]^{[b]} | [%]^{[c]} | [%]^{[d]} |
| 3 | Rh (acac) (CO)₂ | 0 | 11 | 80/20 | 82 | 99/1 | 7 | 100 | 86 |
| 4 | Ir(cod)(acac) | 2 | 13 | 90/10 | 80 | 99/1 | 5 | 98 | 86 |

^{[a]}Reaction conditions unless otherwise specified: substrate = 5.0 mmol, [substrate]₀/[M] = 20,000, [biphephos]₀/[M] = 20, solvent (5 mL): toluene (Rh) or acetonitrile (Ir), 120° C., P = 20 bar CO/H₂ (1:1), Reaction time: 5 h (Rh) or 20 h (Ir).
^{[b]}Distribution (% mol) of substrate remainder, internal alkenes (residual or formed during the reaction), aldehydes (linear or branched) and hydrogenation product such as determined by NMR and GLC analysis.
^{[c]}Conversion of substrate.
^{[d]}Selectivity for hydroformylation products and linear/branched ratio.

1. A method to prepare a fatty nitrile/ester/acid aldehyde comprising the following steps:
1) hydroformylation of a ω-unsaturated fatty nitrite/ester/acid substrate selected from among the compounds of formula CH₂═CH—(CH₂)ᵣ—R, where R is CN or COOR₁, R₁ being H or an alkyl radical having 1 to 4 carbon atoms, r is an integer index such that 1≦r≦13, advantageously 2≦r≦13 and preferably 4≦r≦13, wherein said substrate is reacted with carbon monoxide and dihydrogen under the following conditions:
CO partial pressure of 40 bar or lower, H₂ partial pressure of 40 bar or lower, and the PᵢCO/PᵢH₂ ratio between the respective CO and H₂ partial pressures is in the range of 0.5:1 to 3:1,
temperature in the range of 70 to 150° C.
reaction time of 24 h or less,
in the presence of a catalyst comprising at least one Group VIII metal and at least one bidentate or monodentate ligand,
[substrate]/[metal] molar ratio in the range of 5 000 to 100 000
[ligand]/[metal] molar ratio in the range of 10:1 to 100:1,
so as after the reaction to obtain:
a hydroformylation product comprising at least one fatty nitrile/ester/acid aldehyde of formula: OHC—(CH²)ᵣ₊₂—R, and
an isomerate comprising at least one fatty nitrile/ester/acid isomer with internal unsaturation of which at least 80% of the internal isomer(s) of the isomerate are formed of the ω-1-unsaturated isomer of formula CH₃—CH═CH—(CH₂)ᵣ₋₁—R; followed by:
2) separation and recovery of the fatty nitrile/ester/acid aldehyde and of the isomerate.

2. The method according to claim 1 wherein the ω-unsaturated fatty nitrile/ester/acid substrate meets formula CH₂═CH—(CH₂)ᵣ—R, with R═COOR₁, R₁ being H or an alkyl radical having 1 to 4 carbon atoms.

3. The method according to claim 1, wherein hydroformylation is conducted under CO partial pressure in the range of 10 to 40 bar, under H₂ partial pressure in the range of 5 to 20 bar, and/or with a PᵢCO/PᵢH₂ ratio between the respective CO and H₂ partial pressures in the range of 1:1 to 3:1.

4. The method according to claim 1, wherein hydroformylation is conducted at a temperature within the range of 100 to 130° C., preferably 100 to 120° C., preferably at a temperature of substantially 120° C.

5. The method according to claim 1, wherein hydroformylation is conducted for a time in the range of 1 to 12 h, preferably in the range of 2 to 6 h, preferably in the range of 3 to 5 h, preferably in the order of 4 h.

6. The method according to claim 1, wherein the ligand of the catalyst is a bidentate ligand, advantageously a chelating diphosphine selected from among Dppm, Dppe, Dppb, Xantphos and/or BiPhePhos, preferably selected from among Xantphos and/or BiPhePhos, and is further preferably BiPhePhos.

7. The method according to claim 1, wherein the metal of the catalyst is provided in the form of a precursor comprising said metal and at least one compound selected from among acetylacetonates, carbonyl compounds, cyclooctadienes, chlorine, and mixtures thereof.

8. The method according to claim 7 wherein the hydroformylation catalyst comprises rhodium, preferably provided by a precursor such as Rh(acac)(CO)₂, ruthenium, preferably provided by a precursor such as Ru₃(CO)₁₂, where acac is an acetylacetonate ligand and CO is a carbonyl ligand, and/or iridium, preferably provided by a precursor such as Ir(COD)Cl where COD is a 1,5-cyclooctadiene ligand and Cl is a chlorine ligand, preferably it comprises iridium.

9. The method according to claim 6, wherein hydroformylation is catalyzed by a system selected from among: Rh-Xantphos, Rh-BiPhePhos, Ir-Xantphos, Ir-BiPhePhos and the mixtures thereof.

10. The method according to claim 1, wherein the [substrate]/[metal] molar ratio is in the range of 5000 to 50 000.

11. The method according to claim 7, wherein the [ligand]/[metal] molar ratio is in the range of 20:1 to 100:1, preferably 40:1 to 100:1.

12. The method according to claim 1, wherein hydroformylation is performed using a sufficient amount of solvent to solubilize at least part of the catalyst, preferably in an amount of less than 1%, preferably less than 1/1000 relative to the ω-unsaturated fatty nitrile/ester/acid reactant.

13. The method according to claim 1, wherein the hydroformylation step comprises recycling of the hydroformylation catalyst, optionally completed by the providing of new catalyst and/or ligand at a subsequent hydroformylation cycle.

14. The method according to claim 1, further comprising, prior to the hydroformylation step, a step to pre-treat the substrate, this pre-treatment being performed for example by distillation of the substrate followed by purification via adsorption of the substrate using alumina.

15. The method according to claim 1, further comprising a step:
to separate and recover the isomers from the isomerate, and/or
to convert at least one isomer of the isomerate to isomer derivative(s), in particular by conversion of one or more isomer functions to an acid, aldehyde, alcohol and/or amine function, and/or by reaction(s) of the internal double bond of isomer(s), in particular hydrogenation, epoxidation and/or polymerization.

16. The method according to claim 1 further comprising:
2′) oxidation step in the presence of dioxygen during which the nitrile/ester/acid-aldehyde obtained at step 1) is converted to fatty nitrile/ester/acid acid of formula HOOC—(CH₂)ᵣ₊₂—R, or
2″) a reduction step during which the nitrile/ester/acid aldehyde obtained at step 1) is converted to fatty nitrile/ester/acid alcohol of formula HOC—(CH₂)ᵣ₊₂—R, or to an amino alcohol of formula HOC—(CH₂)ᵣ₊₃—NH₂ for nitrile.

17. The method according to claim 16 wherein the oxidation step is implemented by dispersing &oxygen or a gaseous mixture containing dioxygen in the product resulting from hydroformylation.

18. The method according to claim 16, wherein the oxidation step is implemented without the addition of solvent and/or without the addition of dioxygen-activating catalyst.

19. The method according to claim 16, wherein the oxidation step is implemented under dioxygen partial pressure ranging from 0.2 bar to 50 bar, in particular 1 bar to 20 bar, preferably 1 to 5 bar.

20. The method according to claim 16, wherein the dioxygen is continuously injected into the reaction medium, preferably in the form of a stream of air or oxygen, preferably injected in excess relative to the stoichiometry of the oxidation reaction.

21. The method according to claim 16, wherein the molar ratio of dioxygen relative to the product derived from the hydroformylation step is in he range of 3:2 to 100:2.

22. The method according to claim 16, wherein oxidation is conducted at a temperature in the range of 0° C. to 100° C., preferably 20° C. to 100° C., more preferably 30° C. to 90° C., further preferably 40° C. to 80° C., optionally with two consecutive temperature holds at increasing temperature.

23. The method according to claim 16 also comprising:
3′) a reduction step during which the nitrile acid obtained at step 2′) is converted to ω-amino acid of formula HOOC—(CH₂)ᵣ₊₃—NH₂ with regard to a nitrile acid; or
3″) a hydrolysis step during which the ester acid obtained at step 2′) is converted to diacid of formula HOOC—(CH₂)ᵣ₊₂—COOH with regard to an ester acid.

24. The method according to claim 1, further comprising a polymer synthesis step, in particular a polyamide, by polymerization using the ω-amino acid or diacid obtained at 3′); or polyester, by polymerization using the ester alcohol obtained at step 2″).
